(19) United States
(12) Patent Application Publication
(10) Pub. No.: US 2025/0366875 A1
(43) Pub. Date: Dec. 4, 2025

(54) IMPLANT AND MEDICAL ASSEMBLY FOR THE INSERTION OF SUCH AN IMPLANT INTO A CAVITY OF A HUMAN OR ANIMAL BODY

(71) Applicant: DIANOSIC, STRASBOURG (FR)

(72) Inventors: David BAAS, STRASBOURG (FR); Sarah ZAHOUANI, STRASBOURG (FR)

(21) Appl. No.: 18/881,597

(22) PCT Filed: Jul. 6, 2023
(86) PCT No.: PCT/IB2023/056998

§ 371 Date: Jan. 6, 2025

(30) Foreign Application Priority Data
Jul. 7, 2022 (FR) 2206991

(51) Int. Cl. A61B 17/24; A61B 17/00

(52) U.S. Cl. CPC A61B 17/24; A61B 2017/00867

(57) ABSTRACT

An implant for medical use, for introduction into a cavity in a human or animal body, includes at least one wall formed of an elastic structure intended to surround a biological protuberance present in the cavity in order to fix the implant around the biological protuberance. This wall is essentially cylindrical in shape and formed over an entire cylindrical lateral surface of the implant except in at least one limited angular portion of its circumference, thus imparting radial elasticity to the cylindrical lateral surface of the implant.

The present invention relates to an implant for medical use intended to be introduced into a cavity of a human or animal body. It also concerns an assembly for medical use comprising such an implant and a device designed for its insertion and deployment into a cavity of a human or animal body.

The invention applies more particularly to an implant comprising at least one wall formed of an elastic structure intended to surround a biological protuberance present in the cavity in order to fix the implant around the biological protuberance. It should be noted that such a device remains implantable, and can therefore always be referred to as an “implant”, even if it is not intended to pass through or be introduced into any biological tissue. It should also be noted that the elasticity of the structure is not necessarily correlated with the elasticity of its constituent material. In a manner known per se, it is possible to obtain a structure with elastic properties conferred by its configuration even if the constituent material is weakly, or even very weakly, elastic.

Such an implant is, for example, the subject matter of patent document WO 2019/025695 A1. In this document, the implant comprises at least one clip comprising two flat walls intended more precisely to grip between them a biological protuberance, in particular the middle concha and/or the inferior concha of a human body nasal cavity. Each flat wall comprises an elastic wire-structure element with a number of folds on itself to enable the wall to be laterally contracted prior to insertion of the implant when the latter is introduced into a syringe-shaped insertion and deployment device, and then laterally deployed inside the nasal cavity when exiting this syringe. This enables the implant to achieve large lateral dimensions for stable, durable fixation around the biological protuberance, while allowing introduction into the cavity through a narrow opening.

However, this flat clip configuration is rather complex to manufacture industrially. In addition, the mechanical hold on each biological protuberance concerned is not ideal, and it is therefore advantageous to provide front and rear stabilizers, which makes manufacturing even more complex. Furthermore, the overall shape of the implant, which tends to exert pressure on each biological protuberance it grips, is not anatomically optimal.

It may thus be desired to provide an implant for medical use which makes it possible to overcome at least part of the above-mentioned problems and constraints.

It is thus proposed an implant for medical use intended to be introduced into a cavity of a human or animal body, comprising at least one wall formed of an elastic structure intended to surround a biological protuberance present in the cavity in order to fix the implant around the biological protuberance, wherein said at least one wall is of essentially cylindrical shape and formed over an entire cylindrical lateral surface of the implant except in at least one limited angular portion of its circumference, thus conferring radial elasticity to the cylindrical lateral surface of the implant.

Thus, the cylindrical shape of the implant is easy to manufacture industrially, given the materials generally used for medical purposes. It is further more anatomical, particularly when the biological protuberance in question is the middle or inferior concha of a nasal cavity, since its radial elasticity enables it to be held stably against surrounding anatomical walls in the deployed configuration, while the limited, wall-free angular portion which forms a notch or a complete opening in the direction of the cylinder axis enables it to be positioned around the protuberance by sliding along this limited angular portion. The radial elasticity of such an implant further enables it to be precisely positioned by means of an insertion and deployment device, into which it can be inserted under elastic stress in a contracted configuration.

Optionally, said at least one wall is formed over the entire cylindrical lateral surface of the implant except over the whole length of said at least one limited angular portion of its circumference.

Also optionally, the implant may comprise:
a distal end wherein said at least one limited angular portion is open and free for possible sliding engagement of the biological protuberance within the implant from that distal end; and
a proximal end having an abutment for limiting the possible engagement of the biological protuberance inside the implant up to this abutment.

Also optionally, the implant may comprise a plurality of elongate spatulas, in particular four elongate spatulas, extending from said at least one wall at the distal end of the implant and being folded inwardly of said at least one wall.

Also optionally, the implant may comprise a plurality of extensions extending from the wall at the proximal end of the implant and shaped to form an abutment base for the biological protuberance when the latter is engaged in the implant over the entire length of the limited angular portion, in particular:
three extensions, one of which is ovoid and two of which are kidney-shaped, bent at or near right angles to the inside of said at least one wall; or
two hook-shaped extensions the bases of which are symmetrically arranged, in accordance with a plane of symmetry of the implant centered in the limited angular portion, and the free hook ends of which meet in the limited angular portion.

Also optionally, the implant may comprise a single perforated wall of radially elastic structure with a solid perimeter frame, this single wall being formed over the entire cylindrical lateral surface of the implant except in a single limited angular portion of its circumference.

Also optionally, the implant may comprise a single wall of radially elastic diamond mesh structure, this single wall being formed over the entire cylindrical lateral surface of the implant except in a single limited angular portion of its circumference.

Also optionally, the implant may comprise two semi-cylindrical walls with a radially elastic structural element having undulations so that the structural element is folded several times on itself, these two walls being formed opposite each other over the entire cylindrical lateral surface of the implant except in two limited angular portions of its circumference and diametrically opposed between these two walls.

Also proposed is an assembly for medical use for introducing an implant into a cavity of a human or animal body, comprising:
an implant for medical use according to the invention; and
a device for inserting and deploying the implant for medical use into a cavity of a human or animal body;
   wherein the insertion and deployment device comprise a chamber for receiving and radially contracting the implant so as to coincide a central axis of this radially contracted configuration of the implant with an axis of insertion and deployment of the implant into the cavity of a human or animal body through a distal end of the insertion device.

Optionally, such an assembly for medical use may comprise an implant crimping element with an implant retaining ring in its radially contracted configuration, the retaining ring being slidably mounted around the implant receiving chamber, between a retracted position around a proximal or distal wall of the implant receiving chamber enabling the chamber to be opened, and a crimping position around a central portion of the implant receiving chamber ensuring the chamber's at least partial closure.

The invention will be better understood using the following description, given only by way of example and made with reference to the appended drawings wherein:

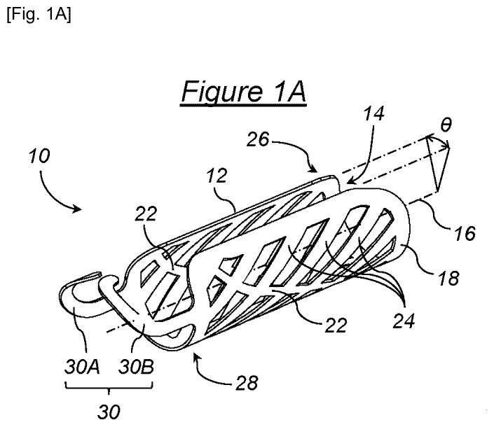

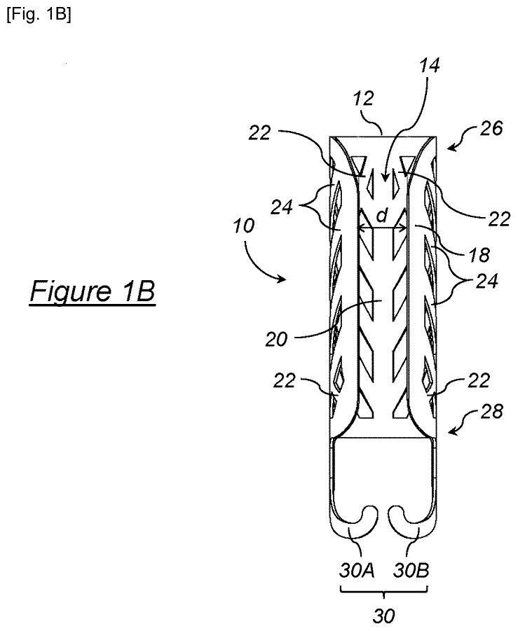

FIG. 1A schematically shows a perspective view of the general structure of an implant for medical use according to a first embodiment of the invention,

FIG. 1B shows a schematic top view of the implant for medical use shown in FIG. 1,

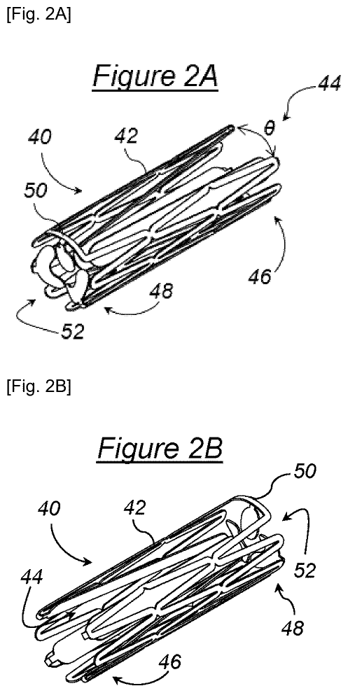

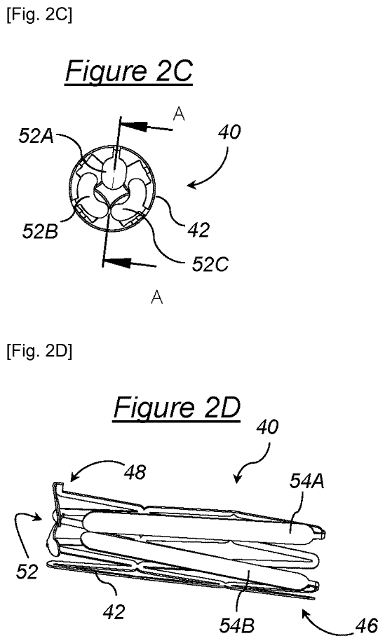

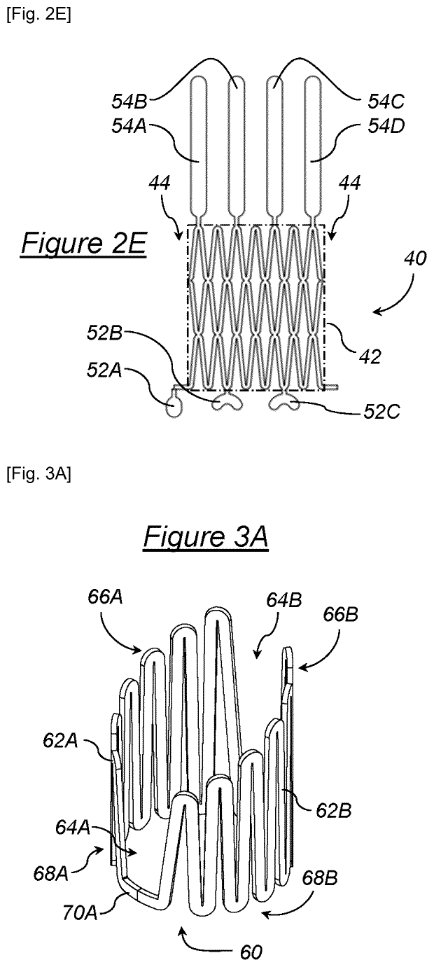

FIG. 2A schematically shows a first perspective view of the general structure of an implant for medical use according to a second embodiment of the invention,

FIG. 2B schematically shows the implant for medical use shown in FIG. 2A according to a second perspective,

FIG. 2C schematically shows a proximal front view of the implant for medical use shown in FIG. 2A,

FIG. 2D shows a schematic cross-section of the implant for medical use shown in FIG. 2A,

FIG. 2E schematically shows a flattened, deployed and open view of the implant for medical use shown in FIG. 2A,

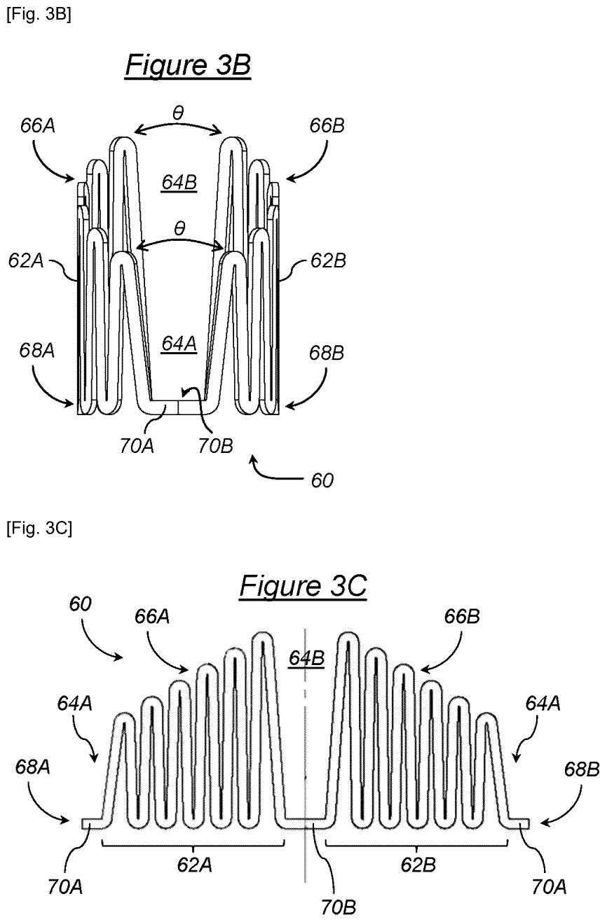

FIG. 3A schematically shows a perspective view of the general structure of an implant for medical use according to a third embodiment of the invention,

FIG. 3B schematically shows a top view of the implant for medical use shown in FIG. 3A,

FIG. 3C schematically shows a flattened, deployed and open view of the implant for medical use shown in FIG. 3A,

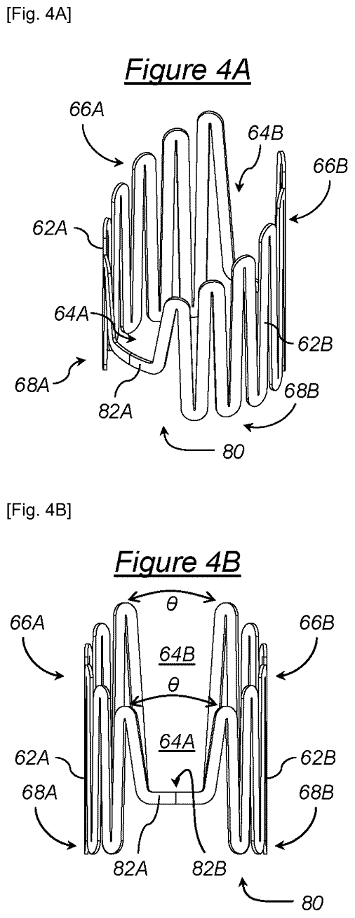

FIG. 4A schematically shows a perspective view of the general structure of an implant for medical use according to a fourth embodiment of the invention,

FIG. 4B schematically shows a top view of the implant for medical use shown in FIG. 4A,

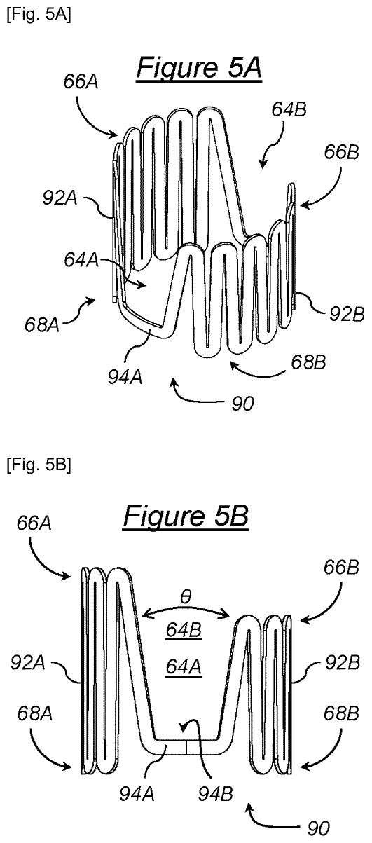

FIG. 5A schematically shows a perspective view of the general structure of an implant for medical use according to a fifth embodiment of the invention,

FIG. 5B schematically shows a top view of the implant for medical use shown in FIG. 5A,

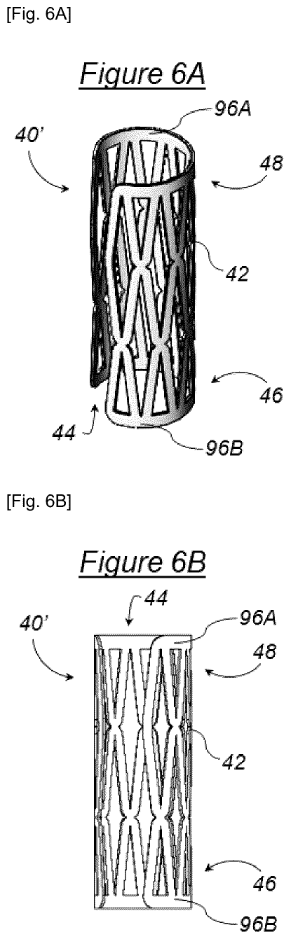

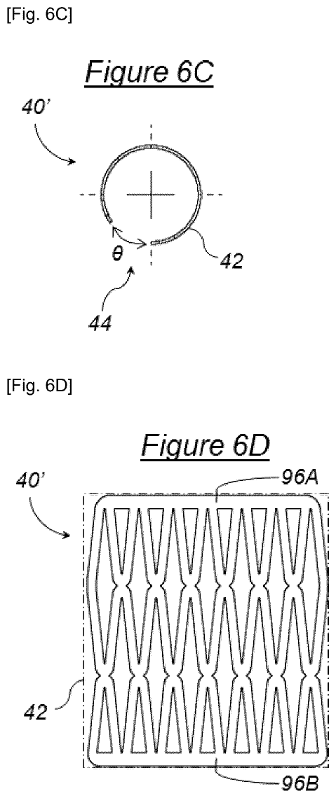

FIG. 6A schematically shows a perspective view of the general structure of an implant for medical use according to a sixth embodiment of the invention,

FIG. 6B schematically shows a top view of the implant for medical use shown in FIG. 6A,

FIG. 6C schematically shows a proximal or distal front view of the implant for medical use shown in FIG. 6A,

FIG. 6D schematically shows a flattened, deployed and open view of the implant for medical use shown in FIG. 6A,

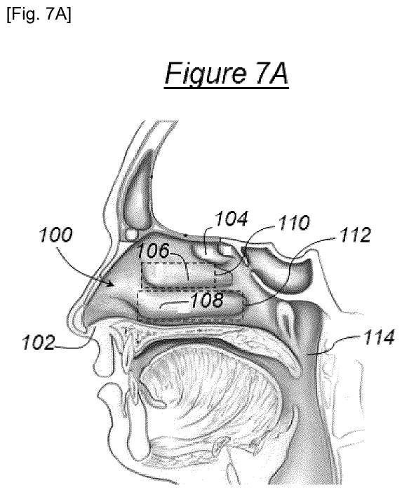

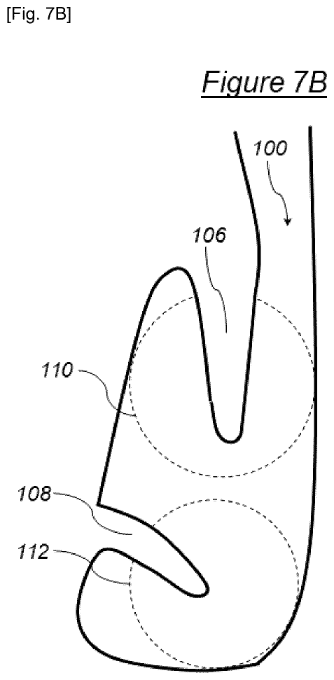

FIG. 7A shows a human nasal cavity in sagittal cross-section, schematically illustrating arrangements of implants for medical use according to the invention,

FIG. 7B schematically shows a human nasal cavity in frontal cross-section, illustrating arrangements of implants for medical use according to the invention,

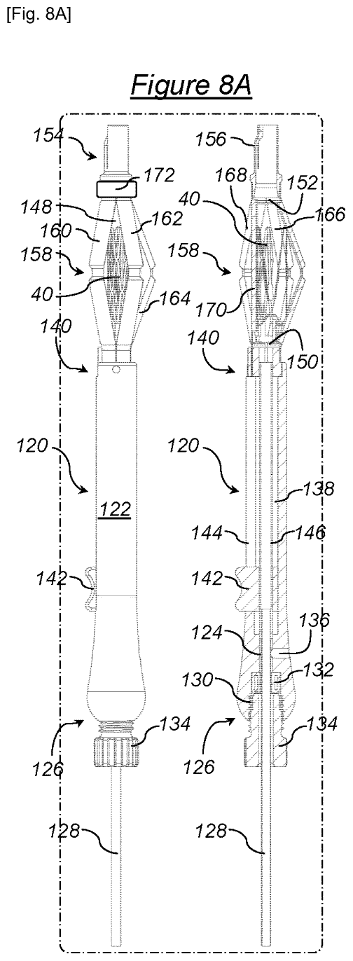

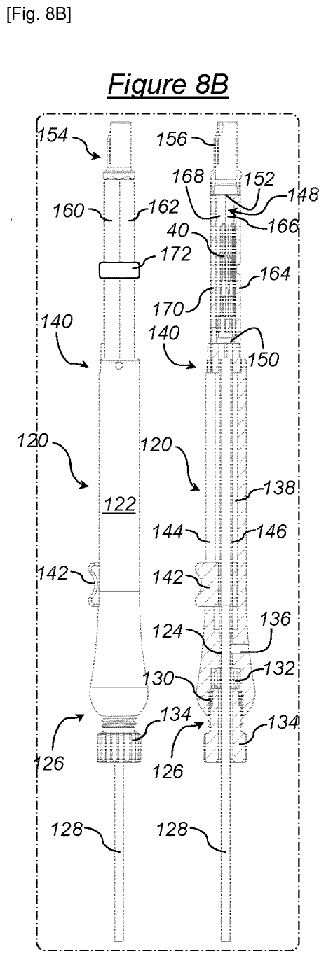

FIG. 8A schematically shows a cross-sectional side view of a medical assembly according to an embodiment of the invention, in a first open configuration,

FIG. 8B shows a cross-sectional side view of the medical assembly shown in FIG. 8A in a second closed configuration,

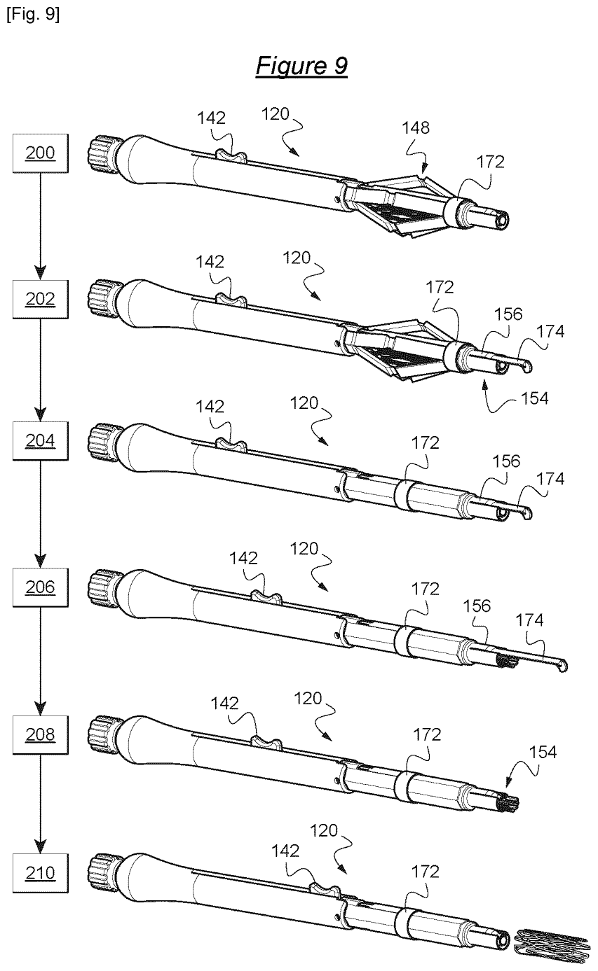

FIG. 9 illustrates the successive steps of a method for inserting and deploying an implant for medical use into a cavity of a human or animal body, using the assembly of FIGS. 8A and 8B.

The implant for medical use 10 schematically shown in perspective on FIG. 1A and in top view on FIG. 1B comprises a single, essentially cylindrical, perforated wall 12 of elastic, more precisely flexible, structure, intended to surround a biological protuberance present into a cavity of a human or animal body in order to fix the implant 10 around this biological protuberance. According to the overall principles of the present invention, this wall 12 is formed over an entire cylindrical lateral surface of the implant 10 except in at least one limited angular portion 14 of its circumference, thus creating a longitudinal opening in the overall tubular shape assumed by the implant 10. This particular shape of wall 12 confers radial elasticity in contraction and expansion to the cylindrical lateral surface of implant 10. More precisely, in this first possible embodiment, wall 12 is formed over the entire cylindrical lateral surface of implant 10 except for the entire length of a single limited angular portion 14. The latter can be defined angularly, i.e. according to an angle θ identified in FIG. 1A, from a central longitudinal axis 16 of implant 10, or by its width, i.e. according to a linear distance d identified in FIG. 1B or a corresponding arc length.

The radial elasticity of implant 10 is achieved, on the one hand, by its perforated configuration and possibly, on the other hand, by the material constituting its wall 12.

As far as the constituent material is concerned, it must be approved for medical use of implantation into a cavity of a human or animal body, as is the case, for example, with nickel-titanium. Advantageously, it is also resorbable, i.e. biodegradable within a predetermined period of time. This predetermined period of time can be defined according to its use, in particular so that the implant does not need to be removed after insertion. But it can also be a simple safety feature, whereby the implant can still be removed after a desired treatment period that is shorter than this predetermined time period. This may be a resorbable polymer matrix, for example a biodegradable polyester matrix, more or less flexible, in particular polycaprolactone (PCL) and/or polylactic acid (PLA).

With regard to the perforated, i.e. openwork, configuration, that of the wall 12 of implant 10 is suitable for a rather flexible, albeit slightly elastic, constituent material such as PCL, i.e. with potentially greater radial elasticity than PLA. The result is a solid perimeter frame 18 surrounding wall 12, suitable for limiting this radial elasticity, and a plurality of solid ribs 20, 22, 24 formed inside this frame 18. The ribs are sufficiently wide to ensure that the essentially cylindrical shape of wall 12 is maintained. A first rib 20, diametrically opposed to the longitudinal opening formed in the limited angular portion 14, extends longitudinally parallel to the latter along the entire length of wall 12 so as to configure it into two contiguous perforated half-walls. Two further ribs 22 extend along a first diameter of each of the two half-walls, from the first rib 20 at a distal end 26 of wall 12 to the vicinity of the limited angular portion 14 at a proximal end 28 of wall 12. A plurality of further ribs 24 extend into each half-wall parallel to its second diameter, for example six further ribs 24 per half-wall in the example shown in FIGS. 1A and 1B.

Thanks to the longitudinal opening created in the implant 10 by the limited angular portion 14, the distal end 26 is open and free in this limited angular portion 14 for possible sliding engagement of a biological protuberance inside the implant 10 from this distal end 26. On the other hand, the proximal end 28 has an abutment 30 for limiting the possible engagement of the biological protuberance inside the implant 10 up to this abutment 30.

More precisely, and in the non-limiting example shown in FIGS. 1A and 1B, abutment 30 is formed by two extensions 30A and 30B which extend from the proximal end 28 of wall 12 and are shaped so as to form an abutment base for the biological protuberance when the latter is engaged in implant 10 over the entire length of the limited angular portion 14. Even more precisely, the two extensions 30A and 30B are in the form of hooks whose bases integral with wall 12 are symmetrically arranged, in accordance with the plane of symmetry of implant 10 centered on limited angular portion 14, on either side of the longitudinal opening created in implant 10 by limited angular portion 14 and the free hook ends of which meet in limited angular portion 14 at a distance from proximal end 28.

In terms of dimensions, the implant 10 is, for example, approximately 10 mm in diameter (in radially extended configuration) for a wall 12 length of approximately 30 mm beyond the proximal end 28 of which the abutment 30 extends for a further approximately 10 mm. The longitudinal opening created by the limited angular portion 14 into which wall 12 does not extend is, for example, of linear width d=4.8 mm, corresponding to an opening angle θ of between π/4 and π/3 rad. More generally, an opening angle θ less than or equal to π/2 rad enables the biological protuberance to be inserted and the implant 10 to be held in position. The two hook ends of abutment 30 come together up to approximately 1.1 mm apart. The implant 10 as thus dimensioned and designed is suitable for placement around an inferior nasal cavity concha of the human body, for the treatment of pathologies such as rhinitis. It should be noted that the constituent material and dimensions of implant 10 can be modified for other applications, such as placement around a middle nasal cavity concha or other protuberance, for an animal, for the treatment of sinusitis, etc.

The implant for medical use 40, shown schematically in perspective on FIGS. 2A, 2B, in proximal front view on FIG. 2C, in cross-section on FIG. 2D according to a section A-A indicated on FIG. 2C, and in flattened, deployed and open view on FIG. 2E, comprises a single, essentially cylindrical wall 42 with a radially elastic, more precisely even extensible, diamond mesh structure intended to surround a biological protuberance present into a cavity of a human or animal body in order to fix the implant 40 around this biological protuberance. It should be noted that FIG. 2E is a theoretical representation, since flattened, deployed and open whereas the implant 40 is cylindrical and laterally closed, but this representation clarifies its structure.

The essentially cylindrical shape of wall 42 provides the implant 40 with a radially elastic, or more precisely, expandable, cylindrical lateral surface, thanks to the diamond meshes. In accordance with the general principles of the present invention, this wall 42 is formed over the entire cylindrical lateral surface of the implant 40 except in at least one limited angular portion 44 of its circumference, thus creating a longitudinal opening in the generally tubular shape assumed by the implant 40. More precisely, in this second possible embodiment, wall 42 is formed over the entire cylindrical lateral surface of implant 40 except for the entire length of a single limited angular portion 44. The latter can be defined angularly, i.e. according to an angle θ similar to that identified in FIG. 1A, from a central longitudinal axis (not illustrated) of implant 40, or by its width, i.e. according to a linear distance similar to that identified in FIG. 1B or a corresponding arc length.

As before, the radial elasticity of the implant 40 is achieved, on one hand, by its diamond mesh configuration and, possibly, on the other hand, by the material constituting its wall 42.

As before, the constituent material may be a resorbable polymer, such as a biodegradable polyester more or less flexible, in particular polycaprolactone (PCL) or polylactic acid (PLA). It can also be nickel-titanium or any other biologically compatible material.

With regard to the diamond mesh configuration, that of the wall 42 of the implant 40 is suitable for a constituent material with a certain rigidity, such as PLA, which is less deformable than PCL, i.e. with potentially limited elasticity and radial extensibility, although PCL is also entirely feasible. As a result, there is no perimeter frame for wall 42 in this embodiment.

Thanks to the longitudinal opening created in the implant 40 by the limited angular portion 44, the distal end 46 of the wall 42 clearly visible in FIG. 2B is open and free in this limited angular portion 44 for possible sliding engagement of a biological protuberance inside the implant 40 from this distal end 46. On the other hand, the proximal end 48 of the wall 42, clearly visible in FIG. 2A, has an abutment 50 for limiting the possible engagement of the biological protuberance inside the implant 40 along the entire length of the limited angular portion 44 up to this abutment 50.

More precisely, the abutment 50 consists at least of a rod extending circumferentially to the proximal end 48. This rod is, for example, an extension of the diamond mesh that forms wall 42. It can be supplemented by a base 52 for abutment 50, clearly visible in FIGS. 2A, 2C and 2E, itself formed, for example, by three extensions 52A, 52B, 52C, one of which is ovoid (52A) and two of which are kidney-shaped (52B, 52C), foldable at right angles, or close to right angles, towards the inside of wall 42 to form this base 52. These three extensions 52A, 52B, 52C also extend from the diamond mesh at the proximal end 48 of wall 42, as shown in FIG. 2E.

The implant 40 further optionally comprises a number of elongated spatulas, in particular four elongated spatulas 54A, 54B, 54C and 54D, extending from wall 42 at its distal end 46 and folded inwards towards wall 42. These are clearly visible in FIGS. 2D and 2E. When the implant 40 is arranged around the biological protuberance and radially deployed to make contact with the surrounding biological walls, they enable internal contact between the implant 40 and the biological protuberance to be maintained.

In terms of dimensions, implant 40 can be similar to implant 10, for placement around an inferior nasal cavity concha of human body and for the treatment of pathologies such as rhinitis. The longitudinal opening created by the limited angular portion 44 wherein the wall 42 does not extend is, for example, of opening angle θ of about 4π/9 rad +/−10%. More generally, an opening angle θ less than or equal to π/2 rad allows insertion of the biological protuberance and retention of the implant 40 in position. The constituent material and dimensions of implant 10 can be modified for other applications, such as placement around a nasal cavity middle concha or other protuberance, for an animal, for the treatment of sinusitis, etc.

The implant for medical use 60, shown schematically in perspective view in FIG. 3A, in top view in FIG. 3B and in flattened, deployed and open view in FIG. 3C, comprises two semi-cylindrical walls 62A and 62B with a wired structural element featuring undulations so that the structural element is folded back on itself several times along a variable length, in particular monotonically increasing or decreasing for each of the two semi-cylindrical walls 62A and 62B. In accordance with the general principles of the present invention, the latter are formed opposite each other over the entire cylindrical lateral surface of the implant 60 except in two limited angular portions 64A and 64B of its circumference and diametrically opposed between these two walls, thus creating a longitudinal through opening in the general tubular shape assumed by the implant 60 so as to surround a biological protuberance present into a cavity of a human or animal body in order to fix the implant 60 around this biological protuberance. It should be noted that FIG. 3C is a theoretical representation, since it is flattened, deployed and open, whereas implant 60 is cylindrical and laterally closed, but this representation clarifies its structure.

The essentially cylindrical shape of the two semi-cylindrical walls 62A and 62B gives the implant 60 a radially elastic, or more precisely, extensible cylindrical lateral surface, thanks to the multiple folding of their wire structure. More precisely, in this third possible embodiment, the two semi-cylindrical walls 62A and 62B are formed over the entire cylindrical lateral surface of the implant 60 except for the entire length of the two limited angular portions 64A and 64B. The latter can be defined angularly, i.e. according to an angle θ similar to that identified in FIG. 1A, from a central longitudinal axis (not illustrated) of implant 60, or by their width, i.e. according to a linear distance similar to that identified in FIG. 1B or a corresponding arc length.

As before, the radial elasticity of the implant 60 is achieved, on the one hand, by the wire-fold configuration of its two walls 62A, 62B and, possibly, on the other hand, by their constituent materials.

As previously, the constituent material may be a resorbable polymer, for example a biodegradable polyester, more or less flexible, in particular polycaprolactone (PCL) or polylactic acid (PLA). It can also be nickel-titanium, which is particularly well suited to a wire structure for medical use. This material is also appreciated for its advantageous properties of cold rigidity, shape memory at human body temperature and super-elasticity. It can also be envisaged in the preceding embodiments.

In the configuration with wire folds, each semi-cylindrical wall 62A or 62B can have, for example, six folds of increasing or decreasing size, as shown in FIG. 3C.

Thanks to the two diametrically opposed longitudinal openings created in the implant 60 by the limited angular portions 64A and 64B, the distal ends 66A and 66B of the two walls 62A and 62B are open and free in these limited angular portions 64A and 64B for possible sliding engagement of a biological protuberance inside the implant 60 from these distal ends 66A and 66B. On the other hand, the proximal ends 68A and 68B of the two walls 62A and 62B each have an abutment 70A, 70B for limiting the possible engagement of the biological protuberance inside the implant 60 over the entire length of the limited angular portions 64A and 64B up to these two abutments 70A, 70B.

More precisely, each abutment 70A, 70B is formed at least by a rod extending circumferentially at the proximal ends 68A and 68B. This rod is, for example, an extension of the folded rod which can form each wall 62A, 62B.

In terms of dimensions, the implant 60 is for example about 22 mm in diameter (in radially extended configuration) for a length of the walls 62A, 62B variable between about 15 and 30 mm, for placement around a nasal cavity middle concha of human body and for the treatment of pathologies such as sinusitis. Each longitudinal opening created by each limited angular portion 64A, 64B into which each wall 62A, 62B does not extend is, for example, of opening angle θ of about π/3 rad +/−10%. More generally, an opening angle θ less than or equal to π/2 rad allows the insertion of the biological protuberance and the retention in position of the implant 60. The constituent material and dimensions of implant 60 can be modified for other applications, such as placement around an inferior nasal cavity concha or other protuberance, for an animal, for the treatment of rhinitis, etc.

A first variant 80 of the implant 60 is shown schematically in perspective on FIG. 4A and in top view on FIG. 4B, in accordance with a fourth embodiment of the invention. Its constitutive elements, which are identical to those of the third embodiment, take up the same references, namely the two semi-cylindrical walls 62A and 62B with a wire structure element, the two limited angular portions 64A and 64B, the two distal ends 66A and 66B of the two walls 64A and 64B, as well as the two proximal ends 68A and 68B of the two walls 64A and 64B.

On the other hand, the two abutments 82A and 82B of implant 80 differ from those 70A, 70B of implant 60 in that they are located not at the proximal ends 68A and 68B of the two walls 64A and 64B, but at a median position relative to the shortest length of the latter, which is located on the side of abutment 82A. This limits the travel of implant 80 along the biological protuberance.

This first variant 80 is also suitable for placement around the middle concha of the nasal cavity of a human or animal body, and for the treatment of pathologies such as sinusitis. It can also be modified for other applications.

A second variant 90 of implant 60 is shown schematically in perspective view in FIG. 5A and in top view in FIG. 5B, in accordance with a fifth embodiment of the invention. Its constitutive elements, which are identical to those of the third embodiment, have the same references, namely the two limited angular portions 64A and 64B, the two distal ends 66A and 66B of the two wire-structure element walls, and the two proximal ends 68A and 68B of these two walls.

On the other hand, the two semi-cylindrical walls 92A and 92B of implant 90, with their wire-like structural elements, differ from those 62A and 62B of implants 60 and 80 in that their lengths are constant and not monotonically increasing or decreasing. More specifically, wall 92A is of large constant length, for example around 30 mm, while wall 92B is of small constant length, for example around 15 mm. The two abutments 94A and 94B of implant 90 also differ from those 70A, 70B of implant 60 and those 82A, 82B of implant 80 by their intermediate arrangement, not quite at the proximal ends 68A and 68B of the two walls 92A and 92B but at an intermediate position between these proximal ends and the median position of the first variant.

This second variant 90 is also suitable for placement around the middle concha of a human or animal nasal cavity and for treating pathologies such as sinusitis. It can also be modified for other applications.

The implant for medical use 40′, shown schematically in perspective on FIG. 6A, in top view on FIG. 6B, in proximal or distal front view on FIG. 6C and in flattened, deployed and open view on FIG. 2D, is a simplified variant of the implant 40 of FIGS. 2A to 2E. It has the same essential features. It differs, however, in the absence of an abutment 50, an abutment base 52 and elongated spatulas 54A, 54B, 54C and 54D. It also differs in that two strips 96A and 96B have been added for two respective proximal and distal reinforcements of its wall 42, in the same spirit as the solid perimeter frame 18 of the embodiment shown in FIGS. 1A and 1B. These two bands 96A, 96B thus extend respectively at the proximal 48 and distal 46 ends in a ring shape over the entire cylindrical lateral surface of the implant 40′ except in the limited angular portion 44 of its circumference.

In terms of material and dimensions, the implant 40′ is similar to the implant 40, for the same uses.

Each of the implants 10, 40, 40′, 60, 80 or 90 is, for example designed to be introduced into a nasal cavity 100 shown in sagittal section in FIG. 7A. The nasal cavity 100 (also referred to as the nasal fossa) extends behind the nostril opening 102 through which these implants can be introduced using a specific insertion and deployment device. The superior 104, middle 106 and inferior 108 conchae act as filters, allowing air and other fluids to circulate freely.

During an ENT surgery affecting the middle concha 106 and/or the inferior concha 108, any of the aforementioned implants can be placed around the middle concha 106 in a cylindrical arrangement 110 shown in dotted lines (in this case, the implant 60, 80 or 90 would be more suitable, but this is not limitative; implants 10, 40 and 40′ could be suitable) and/or around the inferior concha 108 in another cylindrical arrangement 112 also shown in dotted lines (in this case, the implant 10, 40 and 40′ would be more suitable, but this is not limitative either; implants 60, 80 and 90 could be suitable).

FIG. 7B shows a schematic frontal cross-section of the nasal cavity 100. Arrangements 110 and 112 are shown around the middle 106 and inferior 108 conchae in the deployed configuration of the implants, with references 60, 80 or 90 for arrangement 110 and 10, 40 or 40′ for arrangement 112. This shows the support that implants 10, 40, 40′, 60, 80 or 90 can take against the inner walls of nasal cavity 100, while being arranged and fixed around the middle and inferior conchae 106 and 108 by their longitudinal openings. This stable arrangement prevents them from being expelled through the nasal cavity 102, or ingested through the oropharynx 114, as illustrated in FIG. 7A.

A device 120 for inserting and deploying any of the aforementioned implants will now be described with reference to FIGS. 8A and 8B. Other known devices may be envisaged for inserting and deploying any of the aforementioned implants into a cavity of a human or animal body, in particular around a middle or inferior nasal cavity concha. However, device 120 has advantageous technical features for such implants, notably due to their cylindrical shape. However, device 120 is also suitable for other implants than those described above, in particular cylindrical implants without longitudinal opening(s) resulting from an essentially cylindrical wall formed over the entire cylindrical lateral surface of the implant except in at least one limited angular portion of its circumference. In this respect, the object constituted by the insertion device 120 and the object constituted by any one of the aforementioned implants are independent of each other, although advantageously combined into a single assembly for medical use.

A non-limiting embodiment of the insertion and deployment device 120 is shown schematically in a first open configuration in side view and cross-section, respectively in the left and right sections of FIG. 8A.

More specifically, FIG. 8A illustrates a medical assembly for introducing an implant into a cavity of a human or animal body, comprising:
any implant for medical use having a wall formed from an elastic structure, such as for example one of those mentioned above, in particular implant 40 by way of illustration only, and
the insertion and deployment device 120 in its first open configuration.

The device 120 comprises an essentially cylindrical sleeve 122 with a proximal bore 124 for the introduction, through a proximal end 126 of the sleeve 122, of any rod suitable for ENT surgery, such as an endoscope 128. A partially threaded longitudinal hole 130 is drilled in the proximal end 126 around the longitudinal axis of the sleeve 122 to gain access to the bore 124 and also to allow a cylindrical seal 132 to be fitted and a threaded plug 134 to be screwed in. A radial hole 136 is also drilled in sleeve 122 to also access the bore and block the longitudinal travel of endoscope 128 with a pin (not shown) if required.

The proximal bore 124 opens out inside the sleeve 122 into a recess 138 which extends to a distal end 140 of the sleeve 122. This recess 138 contains the base of a finger-operated button 142, sliding along a groove 144 formed longitudinally in the wall of the sleeve 122. It also contains a sliding hollow tube 146, integral with the base of the sliding button 142 and translatable by the latter along the longitudinal axis of the sleeve 122, which is also the axis of insertion and deployment of the implant 40 into a cavity of a human or animal body.

The distal end 140 of sleeve 122 is integral with a chamber 148 which extends it along its longitudinal axis. This chamber 148 receives and radially contracts the implant 40 so that a central axis of this radially contracted configuration of the implant 40 coincides with the longitudinal axis of the sleeve 122. The chamber 148 has a proximal wall 150 for attachment to the distal end 140 of the sleeve 122. On the opposite side, it has a distal wall 152 for attachment to a tubular distal end 154 of the insertion device 120, through which the implant 40 is designed to emerge for insertion and deployment into the cavity of a human or animal body in question. The tubular distal end 154 has two longitudinal notches to form a tongue 156, the free end of which is slightly curved towards the inside of the tube. In this way, this tongue 156 can act as a guide when the implant 40 exits, fitting precisely into its longitudinal opening created by the limited angular portion 44 of its circumference wherein its wall 42 is not formed.

The chamber 148 is surrounded by a crimping element 158 which forms its side walls between its two other proximal 150 and distal 152 walls. More specifically, the crimping element 158 comprises a number of flexible tabs, including five flexible tabs 160, 162, 164, 166 and 168, as well as a rigid tab 170. These six tabs are arranged hexagonally to form a cylindrical chamber 148 with a hexagonal base around the implant 40. Each flexible tab 160, 162, 164, 166 or 168 has two ends of reduced thickness, integral respectively with the proximal wall 150 and the distal wall 152 of chamber 148, and a central portion also comprising a zone of reduced thickness. These zones of reduced thickness ensure its flexibility. The rigid tab 170 is of the same or similar length, but is attached to only one of the two walls, proximal 150 and distal 152, and slides with guidance in the other, to allow the flexible tabs to spread apart without twisting, thanks to their zones of reduced thickness, and to open the chamber 148 to facilitate insertion of the implant 40 into this chamber. It is in this open configuration of chamber 148 that device 120 is illustrated in FIG. 8A.

Advantageously, the longitudinal opening of the implant 40 created by the limited angular portion 44 of its circumference, wherein its wall 42 is not formed, is placed against the rigid tab 170. This rigid tab 170 forms a reference point for angularly positioning the implant 40 around the longitudinal axis of the device 120. It therefore extends in a plane parallel to that of the base of tab 156. It also provides a certain hold for the crimping element 158.

Alternatively, the flexible crimping tabs can be replaced by equivalent crimping means, such as a braided mesh or stent-like structure.

The crimping element 158 further includes a ring 172 for holding the flexible tabs 160, 162, 164, 166 and 168 in a flattened configuration for closing the chamber 148, to maintain the implant in its radially contracted configuration. This retaining ring 172 is slidably mounted along and around the six tabs 160, 162, 164, 166, 168 and 170.

The left-hand side of FIG. 8A shows it in a retracted position around the distal wall 152 of chamber 148, allowing the flexible tabs 160, 162, 164, 166 and 168 to be spread apart and the implant 40 to be inserted into chamber 148.

The left-hand side of FIG. 8B illustrates it in a crimped position around the central portions of the flexible tabs 160, 162, 164, 166 and 168, by flattening them. In this configuration, chamber 148 is closed and the implant inside is radially contracted so that its central axis coincides with the longitudinal axis of insertion and deployment of device 120. Also in this configuration, the diameter of the contracted implant 40 corresponds to that of the sliding hollow tube 146. Thus, by acting on the sliding button 142, it becomes possible to push the implant 40 thanks to the sliding hollow tube 146 towards the distal end 154 of the device 120 and then outwards, i.e. into the cavity of a human or animal body in question.

A method for inserting and deploying an implant for medical use into a cavity of a human or animal body, using the assembly of FIGS. 8A and 8B, will now be described with reference to FIG. 9.

In a first step 200, the insertion and deployment device 120 is in an open configuration. In other words, its chamber 148 is opened by placing the retaining ring 172 in its retracted position around the distal wall 152 and spreading the flexible tabs 160, 162, 164, 166 and 168. The implant 10, 40, 40′, 60, 80 or 90 is placed in chamber 148, e.g. loaded when the insertion and deployment device 120 is assembled.

In an optional step 202, a strip 174 can be introduced into the insertion and deployment device 120 from its distal end 154 to the proximal end of the implant 10, 40, 40′, 60, 80 or 90, passing under the tongue 156 and along the longitudinal opening of the implant, or one of its longitudinal openings.

In a subsequent step 204, the retaining ring 172 is slid into its crimping position around the central portions of the flexible tabs 160, 162, 164, 166 and 168, so as to flatten them and close the chamber 148 around the then radially contracted implant 10, 40, 40′, 60, 80 or 90.

In a subsequent step 206, the implant 10, 40, 40′, 60, 80 or 90 is pushed towards the distal end 154 of the insertion and deployment device 120 by action on the sliding button 142. The strip 174 helps it to position its longitudinal opening against the tongue 156. It can then be withdrawn.

In a subsequent step 208, the distal end of the implant 10, 40, 40′, 60, 80 or 90 is pushed towards the outlet of the distal end 154 of the insertion and deployment device 120 by acting on the sliding button 142, which is now at mid-travel.

Finally, in a last step 210, when the sliding button 142 has reached the end of its travel, the implant 10, 40, 40′, 60, 80 or 90 is fully introduced into the cavity of a human or animal body in question and can be deployed around a targeted biological protuberance.

It clearly appears that an implant for medical use such as one of those described above is easy to manufacture and practical to use, particularly in ENT surgery, for placement around a biological protuberance located inside a cavity of a human or animal that may be difficult to access.

It is also clear that an insertion and deployment device such as the one described above greatly facilitates the introduction of any of the implants described above, and even other radially elastic cylindrical implants, into a cavity of a human or animal that may be difficult to access for placement around a given biological protuberance.

It should also be noted that the invention is not limited to the embodiments described above. Indeed, it will be apparent to those skilled in the art that various modifications can be made to the embodiments described above, in the light of the teaching just disclosed to them. In the detailed presentation of the invention given above, the terms used should not be interpreted as limiting the invention to the embodiments set out in the present description, but should be interpreted to include all equivalents the anticipation of which is within the reach of those skilled in the art by applying their general knowledge to the implementation of the teaching just disclosed to them.

1. An implant for medical use for introduction into a cavity of a human or animal body, comprising at least one wall formed of an elastic structure intended to surround a biological protuberance present in the cavity in order to fix the implant around the biological protuberance, said at least one wall being essentially cylindrical in shape and formed over an entire cylindrical lateral surface of the implant except in at least one limited angular portion of its circumference, the implant wherein said at least one wall is perforated, made of a structure with a solid perimeter frame surrounding said at least one perforated wall and with a plurality of solid ribs formed inside said solid frame, or made of a radially elastic structure with diamond meshes, or made of a structure with a radially elastic structural element having undulations so that the structural element is folded several times on itself, thus conferring radial elasticity to the cylindrical lateral surface of the implant.

2. The implant for medical use according to claim 1, wherein said at least one wall is formed over the entire cylindrical lateral surface of the implant except over the whole length of said at least one limited angular portion of its circumference.

3. The implant for medical use according to claim 1, comprising:
a distal end wherein said at least one limited angular portion is open and free for possible sliding engagement of the biological protuberance within the implant from that distal end; and
a proximal end having an abutment for limiting the possible engagement of the biological protuberance inside the implant up to said abutment.

4. The implant for medical use according to claim 1, comprising:
a distal end wherein said at least one limited angular portion is open and free for possible sliding engagement of the biological protuberance within the implant from that distal end; and
a plurality of elongate spatulas extending from said at least one wall at the distal end of the implant and bent inwardly of said at least one wall.

5. The implant for medical use according to claim 1, comprising a proximal end having an abutment for limiting possible engagement of the biological protuberance inside the implant up to said abutment and a plurality of extensions extending from the wall at the proximal end of the implant and shaped to form a base of abutment for the biological protuberance when the latter is engaged in the implant over the entire length of the limited angular portion.

6. The implant for medical use according to claim 1, comprising a single perforated wall of radially elastic structure with a solid perimeter frame surrounding the perforated wall and a plurality of solid ribs formed inside said solid frame, said single wall being formed over the entire cylindrical lateral surface of the implant except in a single limited angular portion of its circumference.

7. The implant for medical use according to claim 1, comprising a single wall of radially elastic diamond mesh structure, said single wall being formed over the entire cylindrical lateral surface of the implant except in a single limited angular portion of its circumference.

8. The implant for medical use according to claim 1, comprising two semi-cylindrical walls with a radially elastic structural element having undulations so that the structural element is folded several times on itself, these two walls being formed opposite one another over the entire cylindrical lateral surface of the implant except in two limited angular portions of its circumference and diametrically opposed between these two walls.

9. An assembly for medical use for introducing an implant into a cavity of a human or animal body, comprising:
an implant for medical use according to any of claim 1; and
a device for inserting and deploying the implant for medical use in a cavity of a human or animal body;
wherein the insertion and deployment device comprises a chamber for receiving and radially contracting the implant so as to coincide a central axis of this said radially contracted configuration of the implant with an axis of insertion and deployment of the implant into the human or animal cavity through a distal end of the insertion device.

10. The assembly for medical use according to claim 9, comprising an implant crimping element with an implant retaining ring in its radially contracted configuration, the retaining ring being slidably mounted around the implant receiving chamber, between a retracted position around a proximal or distal wall of the implant receiving chamber enabling the chamber to be opened, and a crimping position around a central portion of the implant receiving chamber, ensuring the chamber's at least partial closure.

11. The implant for medical use according to claim 4, wherein the plurality of elongate spatulas include four elongate spatulas.

12. The implant for medical use according to claim 5, wherein the plurality of extensions include
three extensions, one of which is ovoid and two of which are kidney-shaped, bent at or near right angles to the inside of said at least one wall; or
two hook extensions having bases that are symmetrically arranged, in accordance with a plane of symmetry of the implant centered in the limited angular portion, and the free hook ends of which meet in the limited angular portion.
